(19) United States
(12) Patent Application Publication
(10) Pub. No.: US 2005/0025842 A1
(43) Pub. Date: Feb. 3, 2005

(54) Extraction apparatus

(76) Inventor: Kazuko Kuboyama, Setagaya-ku (JP)

Correspondence Address: LAW OFFICE OF BARRY R LIPSITZ, 755 MAIN STREET, MONROE, CT, 06468, US

(21) Appl. No.: 10/894,718

(22) Filed: Jul. 19, 2004

(30) Foreign Application Priority Data
Jul. 31, 2003 (JP) 2003-283895

(51) Int. Cl. A61K 35/78; A23F 3/00

(52) U.S. Cl. 424/725; 99/279

(57) ABSTRACT

To efficiently and naturally extract a very small amount of effective ingredients present in plant, animal, and mineral matters without deteriorating their functional depositions. An extraction apparatus constituted of an extracting means, a depressurizing means for depressurizing the inside of the extracting means and collecting an airflow from the inside of the extracting means, a condensing means of the airflow supplied from the depressurizing means, and a reserve tank of solution extracted from the condensing means, in which the extracting means is constituted of a water storing part and an extracting part for placing an object to be extracted in the same space as the water storing part, the extracting means and the depressurizing means, the depressurizing means and the condensing means, the condensing means and the reserve tank and the reserve tank and the extracting means are respectively connected through airflow passages, the second extracting means and the second depressurizing means are provided in the airflow passage between the depressurizing means and condensing means, and the second extracting means is constituted of a chamber of an object-to-be-extracted through which an airflow can pass.

### BACKGROUND OF THE INVENTION

1. Technical Field

The present invention relates to a technique for extracting a very small amount of effective ingredients present in a plant, animal, and mineral matter without deteriorating their functional depositions.

2. Description of the Related Art

Medicines used for the traditional Chinese treatment are so-called Chinese medicines and most of the Chinese medicines are crude drugs. The crude drugs are respectively obtained by applying a simple processing to natural drugs and may include western crude drugs and the so-called folk medicines. The Chinese medicines are the crude drugs unique to the traditional Chinese treatment and most of them are plants but some of them are animal matters and mineral matters.

360 to 500 types of Chinese medicines are described in the old documents “Shinnohonsokyo” or “Shinhonsobiyo”. However, Chinese medicines normally used in Japan are 200 to 300 types and particularly frequently used medicines among the Chinese medicines are approximately 100 types. Chinese medicines prescribed in accordance with the traditional Chinese treatment are referred to as “hozai” and the Chinese “hozai” includes decoction referred to as hot solution, powdered medicine referred to as powder-type medicine, pill referred to as ball-type medicine, and ointment referred to as paste-type medicine Internal medicines of the traditional Chinese treatment are mainly the above-described decoction.

The decoction is taken after extracting the effective ingredients of crude drugs into water through brewing. For example, in the case of “Kakkon-to” (decoction of pueraria root and other crude drugs), 4 g of puerariae root, 3 g of ephedra herb, 3 g of cinnamon bark, 3 g of licorice, 3 g of peony root, 3 g of jujube fruits, and 3 g of ginger are put in a container as a daily quantity, and 600 ml of water is then added to the container. The container is brewed in medium heat for approximately 1 hour and reduced into the half quantity. The obtained decoction is taken in warm condition between meals by three times a day.

As explained above, a crude drug is brewed in order to extracting ingredients of the crude drug into a hot solution.

A crude drug not suitable for decoction, that is, when the ingredients may be broken due to high-temperature brewing in the decoction treatment process, the above-described powder-type or ball-type medicine are prescribed. However, various problems occur in taking the above conventional Chinese medicines. For example, when a Chinese medicine is used as the decoction, the above troublesome treatment or taking a considerably lot of quantity is required. Such taking of the medicine may be difficult for a certain type of patient. Moreover, because of the solution medicine, it is inconvenient to carry the medicine and it is very troublesome because it is necessary to brew the medicine every day. On the other hand, in the case of powdered-type or ball-type medicine, a necessary quantity of a crude drug is directly taken. Accordingly, a disadvantage occurs that the patient must take the effective ingredients of the crude drug together with the remainder which is not necessary to be taken.

Regarding to the above described problems, a method of preparing a drug by extracting essence from a crude drug is also used. Concerning the extraction of the essence, a crude drug, a method for brewing the drug and eluting it into a solution, a distillation method, a solution extraction method, and the like are used.

However, these conventional extraction methods have a problem that ingredients contained in objected substances cannot be effectively sampled. That is, an ingredient to be broken by the high temperature or a very small amount of active ingredients which cannot be detected by the conventional analysis technique is contained in a crude drug.

Therefore, when brewing a Korean ginseng and extracting ingredients from the Korean ginseng, or extracting the ingredients in accordance with the distillation method, it is impossible to extract a substance which cannot stand a high temperature.

Moreover, an accurate analysis of ingredients is required in the case of the solution extraction method, accordingly, a problem occurs that, for example, it is impossible to extract a very small amount of unknown substances contained in the Korean ginseng.

Recent developments in the field of botany have also shown that, in addition to currently known ingredients, each variety of plant contains several hundred unknown ingredients, and the indispensable role that these ingredients play with regard to physiological function have gradually revealed. As these substances present only in tiny volumes within plants, it is currently difficult to analyze and isolate them. However, research and development is being carried out into these substances, generally referred to as “PHYTO CHEMICALS”, with the expectation that they will be useful for human physiological function. Also in the research and development, an extraction technique becomes an obstacle and it is difficult to properly extract ingredients contained in plant by the conventional extraction technique.

As a result of this situation, therefore, regardless of whether a specific variety of plant is known or thought to be beneficial, it is often not possible to avail of its benefits at present.

The present invention is made under the background described above.

### SUMMARY OF THE INVENTION

The conventional extraction technique cannot effectively naturally extract a very small amount of effective ingredients present in plant, animal, and mineral matters without deteriorating their functional depositions.

The present invention solves the above problems by constituting an extraction apparatus by a extracting means, a depressurizing means for depressurizing the inside of the extracting means and collecting an airflow from the inside of the extracting means, a condensing means of the airflow supplied from the depressurizing means, and a reserve tank of solution supplied from the condensing means. The extracting means is constituted of a water storing part and an extracting part placing an object to be extracted in the same space as the water storing part, and the extracting means and the depressurizing means, the depressurizing means and the condensing means, the condensing means and the reserve tank, and the reserve tank and the extracting means are connected by airflow passages respectively.

Moreover, in the case of the above extraction apparatus, the extracting means may be provided with a heating means capable of adjusting temperature.

Furthermore, in the case of any one of the above extraction apparatuses, the water storing part of the extracting means may be provided with a water atomizing means.

Furthermore, in the case of any one of the above extraction apparatuses, the depressurizing means may be constituted of a blower provided in the communication passage connecting the extracting means and the condensing means.

Furthermore, in the case of any one of the above extraction apparatuses, the second extracting means and the second depressurizing means are provided in the airflow passage between the depressurizing means and the condensing means and the second extracting means may be constituted of a chamber of an object-to-be-extracted through which an airflow can pass.

Furthermore, in the case of the above extraction apparatus, the second depressurizing means may be constituted of a blower.

The present invention makes it possible to effectively extract ingredients which cannot be conventionally extracted from various substances at a low temperature through the above described constitute and function. That is, it is possible to effectively naturally extract a very small amount of effective ingredients present in plant, animal, and mineral matters without deteriorating their functional depositions.

### BRIEF DESCRIPTION OF THE DRAWINGS

FIG. 1 is a block diagram showing the constitution of the extraction apparatus of the first embodiment;

FIG. 2 is a block diagram showing the constitution of the extraction apparatus of the second embodiment;

FIG. 3 is a perspective view showing the extracting means;

FIG. 4 is a locally cut-out perspective view showing the extracting part (blower) of the extracting means;

FIG. 5 is a perspective view showing the external cylinder of the second extracting means; and

FIGS. 6(a) to 6(c) are perspective views showing the internal cylinder of the second extracting means.

### DETAILED DESCRIPTION OF THE PREFERRED EMBODIMENT

In the first extraction step, atomizing of water is progressed under a depressurized state while the atomized fine particles are made to capture contained ingredients effectively exuded on the surface of a raw material to be extracted in a depressurized atmosphere, and in the second extraction step, the atomized fine particles are made to capture the contained ingredients effectively exuded on the surface of the raw material to be extracted in a further depressurized atmosphere to effectively extract and make it possible to extract ingredients which cannot be easily extracted.

Embodiments

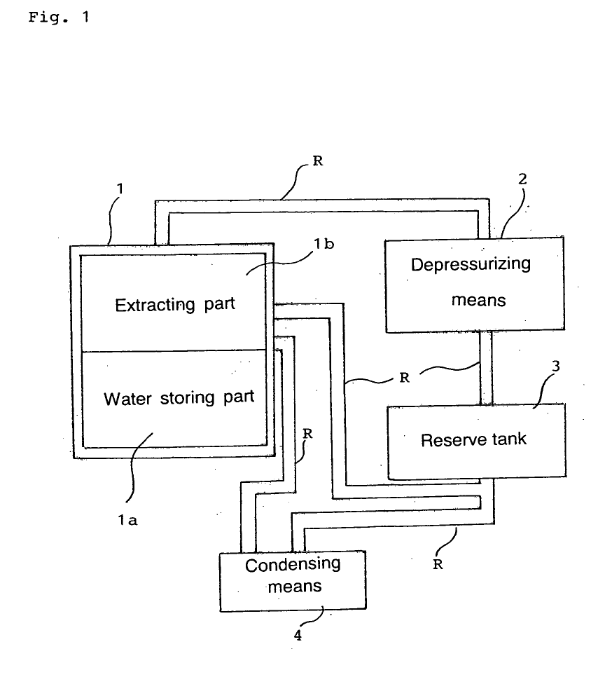

An extraction apparatus according to the present invention is described by referring to the accompanying drawings. FIG. 1 is a block diagram showing a constitution of the first embodiment of the extraction apparatus.

In FIG. 1, symbol 1 denotes a extracting means, 2 denotes a depressurizing means for depressurizing the inside of the extracting means 1 and collecting atomized fine particle airflow from the inside of the extracting means 1, 3 denotes a condensing means of the atomized fine particle airflow circulated from the depressurizing means 2, and 4 denotes a reserve tank of a solution extracted from the condensing means 3. The extracting means 1 and the depressurizing means 2, the depressurizing means 2 and the condensing means 3, the condensing means 3 and the reserve tank 4, and the reserve tank and the extracting means 1 are respectively connected through an airflow passage R and the atomized fine particles generated in the extracting means 1 can circulate through the above means by passing through the airflow passage R.

The extracting means 1 is constituted of a water storing part 1a and an extracting part 1b for placing an object to be extracted in the upper space of the water storing part 1a in the same space as the water storing part 1a so that the atomized fine particles generated in the water storing part 1a contacts with the object to be extracted set in the extracting part 1b to capture the exuded on the surface of the object to be extracted as described later.

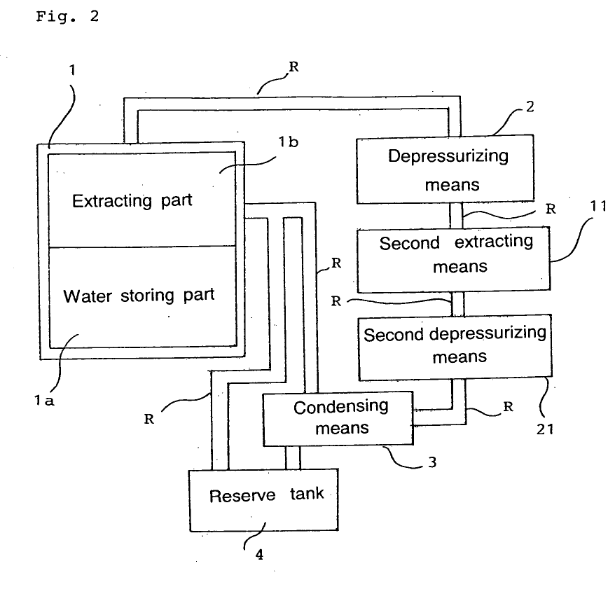

FIG. 2 is a block diagram showing a constitution of the second embodiment of the extraction apparatus.

In FIG. 2, symbol 1 denotes an extracting means, 2 denotes a depressurizing means for depressurizing the inside of the extracting means 1 and collecting a atomized fine particle airflow from the inside of the extracting means 1, 3 denotes a condensing means of the atomized fine particle airflow circulated from the depressurizing means 2, and 4 denotes a reserve tank of a solution extracted from the condensing means 3. The extracting means 1 and the depressurizing means 2, the depressurizing means 2 and the condensing means 3, the condensing means 3 and reserve tank 4, and the reserve tank 4 and extracting means 1 are respectively connected through the airflow passage R so that the atomized fine particles generated in the extracting means 1 can circulate through the above means by passing through the airflow passage R. Thus, the basic constitution of the second embodiment is the same as that of the first embodiment but the second embodiment is different from the first embodiment in that the second extracting means 11 and the second depressurizing means 21 are set between the depressurizing means 2 and the condensing means 3. These second extracting means 11 and the second depressurizing means 21 are connected in series through the airflow passage R between the depressurizing means 2 and the condensing means 3.

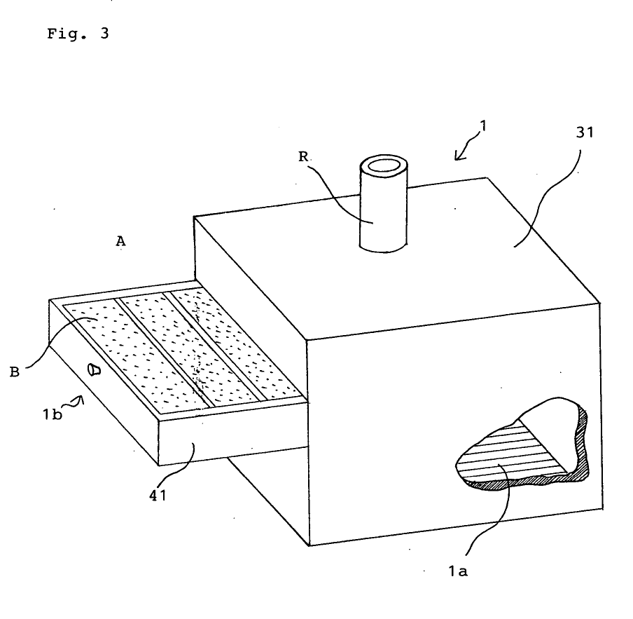

FIG. 3 is a perspective view showing the detail of the extracting means 1 shown in FIGS. 1 and 2. The extracting means 1 has an extracting part 1b set on a casing 31 and a water storing part 1a constituted of the lower part of a casing 1.

Moreover, the extracting part 1b is constituted of a drawer 41 which is set on the casing 31, stores an object B to be extracted which is a raw material and can be moved in the direction of an arrow A.

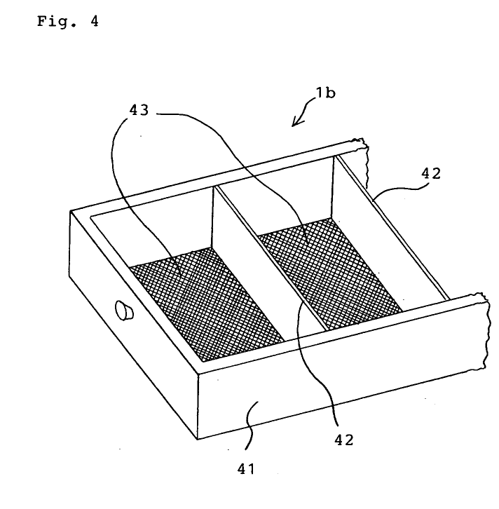

FIG. 4 is a locally-cut-out perspective view showing the detail of the drawer 41. In FIG. 4, symbols 42 and 42 denote rectifying plates set in parallel inside the drawer 41 and these rectifying plates 42 facilitate the movement of atomized fine particles in the raw material rising from the water storing part 1a. Moreover, the bottom of the drawer 41 is constituted of a net body 43 and the atomized fine particles pass through the net body 43, reach and contact with the raw material, and capture the ingredients oozed on the surface of the raw material.

Moreover, the water storing part 1a of the atomizing extraction means 1 is provided with a proper-type heating means capable of adjusting temperature to accelerate the atomization of the water. Furthermore, the water storing part of the extracting means is provided with an ultrasonic oscillator as a means for atomization of the water to accelerate the atomization of the water.

Furthermore, the depressurizing means and the second depressurizing means are respectively constituted of an electric rotating blower provided in the airflow passage.

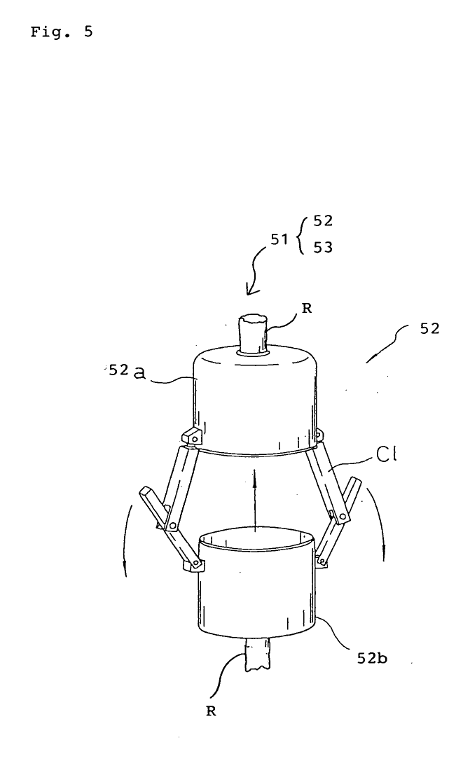

A chamber of an object-to-be-extracted 51 serving as the second extracting means 11 is constituted of an external cylinder and an internal cylinder to be housed in the external cylinder. FIG. 5 is an appearance perspective view showing the external cylinder 52. The external cylinder 52 is constituted of the first external cylinder 52a and the second external cylinder 52b, and the first and second external cylinders 52a and 52b are supported by a clamping device C1 so as to be freely connected and separated and are stainless-steel members respectively having a cylindrical shape of predetermined diameter and depth. A temperature sensor for detecting the temperature on extraction procedure is attached to the lower second external cylinder 52b.

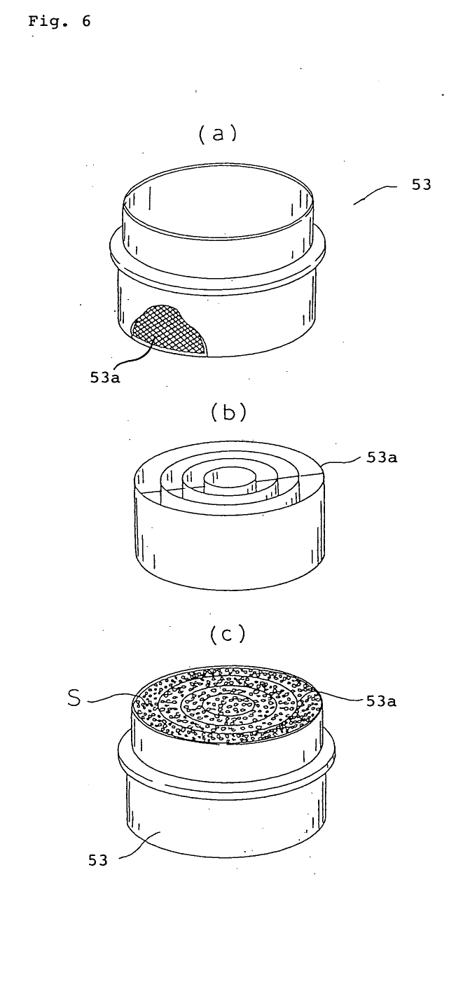

FIGS. 6(a) to 6(c) are exploded perspective views of the internal cylinder 53. As shown in FIG. 6(a), the internal cylinder 53 has dimensions and a shape capable of being fitted to the external cylinder 52 and the bottom of the internal cylinder 53 is provided with a net part 53a for holding a raw material crushed into small pieces. Moreover, FIG. 6(b) is a perspective view showing a guide plate 53a to be fitted into the internal cylinder 53 so as to partition crushed pieces S of a desired raw material such as crude medicine, soy beans, malt, or Korean ginseng in the internal cylinder 53 as shown in FIG. 6. The guide plate 53 has an effect for facilitating the passing to atomized fine particles as described later. It is also allowed to form the partition wall of the guide plate 53 into a spiral shape.

Function of an extraction apparatus are described below in accordance with the above constitution. The raw material used for the embodiment is a Korean ginseng notable as a crude medicine. First, the Korean ginseng crashed into rice-sized grains is charged into the drawer 41 shown in FIGS. 3 and 4. The charged raw material is partitioned by the rectifying plates 42 and 42.

After charging the raw material, the ginseng can be stably held by the drawer 41 covering the ginseng with a net.

Then, the drawer 41 is fitted into the casing 31 as shown in FIG. 3. Moreover, approximately 30 to 50 litters of water is stored in the water storing part 1a shown in FIG. 3. The above quantity of water is always automatically maintained.

When completing the preparation of the water in the water storing part 1a and the Korean ginseng serving as the raw material in the drawer 41, the internal water temperature is set by the heater of the water storing part 1a. If a Korean ginseng is used as a raw material, experience has shown that a set temperature of 85° C. is the most suitable. The temperature of 85° C. is the most suitable temperature to maintain the temperature in the extraction apparatus 2 at 60 to 70° C. as described later.

When the temperature of the water in the water storing part reaches the set temperature of 85° C., the switch for the ultrasonic oscillator is turned on, as does the switch for the blower serving as the depressurizing means 2. An airflow circulates through the extracting means 1, the depressurizing means 2, the condensing means 3, the reserve tank 4, and the blower 2, and the circulatory route formed by the airflow passage R connecting these components.

That is, atomized fine particles of water generated in the water storing part 1a rise through the casing 31 in accordance with the depressurizing action of the blower 2, reaches the drawer 41 together with the airflow, and contacts with the Korean ginseng of the raw material placed inside the drawer 41. Internal effective ingredients are exuded on the surface of the Korean ginseng by depressurizing the inside of the casing 31, and the effective ingredients are captured by the contacted atomized fine particles. The atomized fine particles holding the effective ingredients pass through the airflow passage R and finally reaches the condensing means 3 and are liquefied and instilled into the reserve tank 4.

It is preferable that the temperature of the atomized fine particles in the casing 31 ranges between 60 and 70° C. as described above. For this purpose, the temperature in the casing 31 is always detected by a temperature sensor and the water temperature in the water storing part 1 is controlled in accordance with the detected result of the sensor in order to obtain the preferable temperature.

As described above, the airflow circulates through each component in accordance with the operation of the blower 2 serving as the depressurizing means, however, since the Korean ginseng of the raw material is charged into the extracting part 1b, the airflow rising through the casing 31 suffers a drag therefrom. On the contrary, an object for interrupting passing of the airflow is not present in the circulatory route continuing on the airflow passage R from the casing 31. Therefore, the space in the casing 31 falls in a depressurized state.

When the space in the casing 31 is in the depressurized state, already-known and unknown ingredients contained in the ginseng of the raw material are exuded on the surface of crushed pieces of the ginseng. The various types of the ingredients exuded on the surface of the ginseng pieces are captured by the passing atomized fine particles. As described above, because the internal temperature, specifically the temperature in the drawer 41, is maintained at approximately 65° C., the ingredients contained by the ginseng are extracted into the atomized fine particles without being broken due to heat.

The atomized fine particles containing the effective ingredients of the ginseng pass through the airflow passage R together with the airflow and reach the cooler (not illustrated) of the condensing means 3. The atomized fine particles contacting with the cooler are liquefied and changed into water containing the effective ingredients of the ginseng. The water containing the extracted ingredients of the ginseng is instilled into the reserve tank 4, filtered to remove impurities, and then collected as the water containing the extracted ingredients mainly containing the effective ingredients of the ginseng as the final product.

On the other hand, the atomized fine particles not liquefied by the condensing means 3 are sucked and re-circulated to the casing 31 in which the airflow passage R is depressurized, and then rise and contact with the Korean ginseng in the drawer 41 again to perform an extracting operation. Moreover, the atomized fine particles in the reserve tank 4 are also sucked and re-circulated to the casing 31 in the similar manner as described to perform the extracting operation again.

As described above, when the atomized fine particles circulate through the airflow passage R, the effective ingredients of the ginseng of a raw material is captured in the atomized fine particles and the water containing the extracted ingredients containing the effective ingredients of the ginseng is obtained by liquefying the atomized fine particles. The duration of a single operation of the production apparatus is 1 hour. That is, when the extraction was carried out for one hour in accordance with the embodiment explained above and using approximately 1,800 g of crashed pieces of the ginseng, approximately 3 to 4 litters of water containing the extracted ingredients could be finally produced.

The above extracting operation shows a case of using the extraction apparatus shown in FIG. 1. In the case of the extraction apparatus shown in FIG. 2, after the extraction by the extracting means 1, the extracting operation is further repeated by the second extracting means 11. That is, the atomized fine particles capturing and holding the effective ingredients of the raw material in the extracting part 1b of the extracting means 1 reach the storing chamber of the object-to-be-extracted 51 of the second extracting means 11 constituted of the external cylinder 52 and the internal cylinder 53 in accordance with the sucking action of the blower 2 serving as the depressurizing means. In this case, the atomized fine particles contact with crushed pieces of a desired raw material such as crude drug, soy beans, malt, or Korean ginseng. The effective ingredients of the raw material are exuded on the surface of the crushed pieces S because the inside of the internal cylinder 53 is depressurized by the blower serving as the second depressurizing means, and captured and held by the atomized fine particles. The addition of the second extracting means 11 and the second depressurizing means 21 enables to effectively obtain an extracted solution with high concentration.

In the case of the above embodiment, rice-sized crashed pieces of ginseng is used. However, it is possible to adjust the concentration of effective ingredients contained in the final product by changing the size of crushed pieces. That is, as crushed pieces of ginseng are made smaller, it is possible to obtain the product having a higher concentration. In this case, the production quantity per hour decreases. In contrast, by making the size of the crashed pieces larger, the yield for hour increases, and the ingredient concentration decreases.

In the case of the above embodiment, the guide plate 53a is used for the internal cylinder 53 of the chamber of the object-to-be-extracted 51 in the second extracting means 11. When using the guide plate, the yield of the water containing the extracted ingredients per hour increases by approximately 20% compared to the case of not using the guide plate, but the concentration decreases.

The water containing the extracted ingredients obtained using the extraction apparatus described above is a colorless, transparent, and clean liquid.

In the usage of a Korean ginseng as an decoction in the traditional Chinese treatment, the breaking of the ingredients due to the high-temperature brewing has bee inevitable. However, because the technique of the present invention adopts the extraction at a low temperature, it is possible to effectively capture effective ingredients contained in the ginseng into water. The water containing the extracted ingredients of the ginseng can be taken as it is, or can be used together with the water containing the extracted ingredients which is extracted from the other crude drug in the similar manner as described in accordance with a prescription based on the traditional Chinese treatment.

That is, by blending waters containing the extracted ingredients from various crude drugs, it is possible to use the water similarly to the conventional decoction.

Though examples to be applied to crude drugs in the traditional Chinese treatment are described above, it is a matter of course that raw materials from which ingredients will be extracted are not restricted to the crude drugs. In the case of the past experiments, ingredients are extracted from Chinese crude drugs including a Korean ginseng, soy beans, coffee beans, malt, and koji and trial is repeated for the use of them. In the case of the application of the crude drugs in the conventional Chinese medicine, a troublesome work that a patient individually performs brewing everyday is eliminated. Moreover, because the extraction efficiency is good, it is possible to use raw materials same quantity as ever for more patients.

Moreover, because effective ingredients can be extracted at a low temperature, it is possible to use a crude drug which has been prescribed only as powered medicine or balls in the form of a small quantity of liquid drug and advantages such as easiness of taking and saving of the amount of the crude drugs are obtained.

Furthermore, the present invention makes it possible to take efficiently and directly the effective ingredients of soy beans, molts, and koji, which have been regarded as substances containing the ingredients having positive effect on human body from early times.

1. An extraction apparatus comprising, an extracting means, a depressurizing means for depressurizing the inside of the extracting means and collecting an airflow from the inside of the extracting means, a condensing means of an airflow supplied from the depressurizing means, and a reserve tank of solution extracted from the condensing means, the extracting means is constituted of a water storing part and an extracting part for placing an object to be extracted in the same space as the water storing part, wherein the extracting means and the depressurizing means, the depressurizing means and the condensing means, the condensing means and the reserve tank, the reserve tank and the extracting means are respectively connected through airflow passages.

2. The extraction apparatus according to claim 1, wherein the extracting means is provided with a heating means capable of adjusting temperature.

3. The extraction apparatus according to claim 1, wherein the water storing part of the extracting means is provided with a water atomizing means.

4. The extraction apparatus according to claims 1, wherein the depressurizing means is a blower provided in a communication passage for connecting the extracting means and the condensing means.

5. The extraction apparatus according to claims 1, wherein a second extracting means and a second depressurizing means are provided in an airflow passage between the depressurizing means and the condensing means and the second extracting means is constituted of a chamber of an object-to-be-extracted through which the airflow can pass.

6. The extraction apparatus according to claim 5, wherein the second depressurizing means is a blower.
